(19) United States
(12) Patent Application Publication
(10) Pub. No.: US 2025/0041254 A1
(43) Pub. Date: Feb. 6, 2025

(54) USE OF SODIUM FORMATE IN PREPARATION OF AN ANTI-INFECTIVE DRUG

(71) Applicant: SUN YAT-SEN UNIVERSITY, Guangdong (CN)

(72) Inventors: Hui LI, Guangdong (CN); Bo PENG, Guangdong (CN); Xuanxian PENG, Guangdong (CN); Sufang KUANG, Guangdong (CN)

(73) Assignee: SUN YAT-SEN UNIVERSITY, Guangdong (CN)

(21) Appl. No.: 18/717,426

(22) PCT Filed: Dec. 15, 2022
(86) PCT No.: PCT/CN2022/139358

§ 371 Date: Jun. 7, 2024

(30) Foreign Application Priority Data
Aug. 24, 2022 (CN) 202211021533.9

(51) Int. Cl. A61K 31/19; A61K 31/7048; A61P 31/04

(52) U.S. Cl. CPC A61K 31/19; A61K 31/7048; A61P 31/04

(57) ABSTRACT

The present invention belongs to the technical field of biological medicine, and specifically relates to use of sodium formate in preparation of an anti-infective drug. The present invention has found through research that sodium formate can significantly increase the sensitivity of a bacterium to an antibiotic, so that the antibiotic that is originally ineffective or inefficient against a pathogenic bacterium becomes effective or efficient, thereby killing the bacterium and achieving an anti-infective effect. Sodium formate and an antibiotic are further prepared into an anti-infective composition, so that on the one hand, a significant anti-infective effect can be achieved under the condition of a low-concentration antibiotic, and on the other hand, the decreased use amount of the antibiotic can also significantly reduce the likelihood of a bacterium developing drug resistance; and sodium formate has been widely used in food and medicine with high safety.

### TECHNICAL FIELD

The present invention belongs to the technical field of biological medicine. More specifically, it relates to use of sodium formate in preparation of an anti-infective drug.

### BACKGROUND OF RELATED ART

Bacterial drug resistance has become a major challenge in global public health. Among the many drug-resistant bacteria faced in clinical practice, the most prominent is carbapenem-resistant Gram-negative bacteria, especially carbapenem-resistant Enterobacteriaceae (CRE), which has been increasing rapidly in recent years.

In the face of CRE infection, in addition to developing new drugs, current control measures include: (1) adopting monotherapy, such as polymyxin, tigecycline, fosfomycin, carbapenems, aminoglycosides, etc.; however, monotherapy has its own limitations, and with the widespread clinical application, the drug resistance rate of CRE to drugs such as polymyxin and tigecycline is also on the rise. (2) Performing combination therapy, which combines multiple aspects to treat bacterial infections. For example, the Chinese patent application CN106377527A discloses a method of using open-chain pyridine carboxylic acid derivative H2dedpa in combination with meropenem for anti-CRE, which has good antibacterial effect, but in general, methods of effective antibacterial combination therapy are still lacking. In 2017, the WHO listed CRE as the drug-resistant bacteria that most need new antibacterial drugs. How to diagnose, treat and prevent CRE infection has become the most challenging issue in the current anti-infection field.

### SUMMARY OF THE INVENTION

The technical problem to be solved by the present invention is to overcome the defects and deficiencies that the existing drug-resistant bacterial infections are difficult to treat, and to provide use of sodium formate in preparation of an anti-infective drug.

The purpose of the present invention is to provide an anti-infective composition.

Another purpose of the present invention is to provide an anti-infective drug.

The above purposes of the present invention are achieved through the following technical solutions:

Sodium formate, also known as formic acid sodium salt, and containing two crystalline waters in its crystals, is one of the simplest organic carboxylate salts. Existing technological research has found that sodium formate has multiple uses, for example, in the production of formic acid, oxalic acid, formamide, and sodium hydrosulfite; being used as a catalyst and a stable synthetic agent, etc.; being used as a food preservative in the food industry for soy sauce, vinegar, low-salt pickles, fruit juice, jam, fruit wine, soda, beverage syrup, tobacco, etc.; in the preparation of drugs such as caffeine sodium benzoate sedatives and in the preservation of traditional Chinese medicine pills and syrup in the pharmaceutical industry; and also being used in anti-corrosion and mold prevention for anti-rust paper, latex paint, shoe polish, glue, and fabric; in addition, it can also be used as a mordant in the dye manufacturing industry, a plasticizer in the plastic industry, and a raw material in the perfume industry.

The present invention has found through research that sodium formate can significantly increase the sensitivity of a bacterium to an antibiotic, so that the antibiotic that is originally ineffective or inefficient against a pathogenic bacterium becomes effective or efficient, thereby killing the bacterium and achieving an anti-infective effect. Therefore, the present invention requires the protection of the use of sodium formate in preparation of an anti-infective drug.

Further, the sodium formate in the anti-infective drug enhances sensitivity of a bacterium to an antibiotic.

Preferably, the antibiotic is an aminoglycoside antibiotic.

More preferably, the aminoglycoside antibiotic is one or more of micronomicin, gentamicin, and amikacin.

The above-mentioned antibiotic cannot be used as a limitation on the scope of protection of the present invention. This is because although there are hundreds of types of antibiotics, they can be classified according to their chemical structure and antibacterial mechanism. Similar chemical structures have the same antibacterial mechanism, so they do not need to be verified one by one. Micronomicin, gentamicin, and amikacin are commonly used aminoglycoside antibiotics in clinical practice and have good representativeness. Based on the concept of the present invention, those skilled in the art can easily infer that other clinical aminoglycoside antibiotics can also be applied to the method described in the present invention.

Furthermore, the bacterium is an antibiotic-sensitive or clinically drug-resistant Gram-negative bacterium.

Preferably, the clinically drug-resistant bacterium can be a clinically multidrug-resistant bacterium; preferably, the clinically drug-resistant bacterium is a carbapenem-resistant bacterium, methicillin-resistant bacterium, or the like.

More preferably, the bacteria are antibiotic-sensitive or clinically drug-resistant Escherichia coli, Klebsiella pneumoniae, Pseudomonas aeruginosa, Edwardsiella tarda, Vibrio, or Staphylococcus aureus.

It should be noted that these bacteria are common pathogenic bacteria and their drug-resistant strains are common. At the same time, Escherichia coli and Pseudomonas aeruginosa are type strains for studying bacterial resistance, so these bacteria are good representatives of drug-resistant and non-drug-resistant bacteria. Although in the embodiments of the present invention, the listed bacteria include clinically multidrug-resistant and carbapenem-resistant or carbapenem-sensitive Escherichia coli, Klebsiella pneumoniae, and Pseudomonas aeruginosa, as well as sensitive bacteria for each type of bacteria, clinical Edwardsiella tarda, and Vibrio. Especially, most of the validation experiments of the present invention take clinical multidrug-resistant and carbapenem-resistant Escherichia coli as the research object. However, these bacteria cannot be used as a limitation on the scope of protection of the present invention. This is because: 1) Escherichia coli and Pseudomonas aeruginosa are type strains for studying drug-resistance mechanisms; 2) bacteria can have both drug-resistant and non-drug-resistant states, i.e., there are drug-resistant and non-drug-resistant strains of the same bacteria, while the clinical Escherichia coli of the present invention is in a drug-resistant state, and the addition of sodium formate also improves the sensitivity to antibiotics. Therefore, based on the above principles, it can be inferred from these strains that more strains are also suitable for the concept of the present invention.

Additionally, the present invention provides an anti-infective composition, the composition including sodium formate and an aminoglycoside antibiotic. Combining sodium formate with the antibiotic as an anti-infective composition can efficiently kill a bacterium by a synergistic effect under the condition of a low-concentration antibiotic, to achieve good anti-infective effects.

Preferably, the aminoglycoside antibiotics are one or more of micronomicin, gentamicin, and amikacin.

Preferably, a mass ratio of the sodium formate to the antibiotic is (0.088-90.67): 1.

In addition, the present invention also requires the protection of an anti-infective drug, the anti-infective drug including the anti-infective composition.

In addition to having an effect on the treatment of bacterial infections in mice and humans, the drug can also be applied in the field of veterinary treatment to treat bacterial infections in organisms such as pets, livestock and poultry, such as mammals, rodents, and the like. Other examples of animals include horses, dogs, cats, and the like.

Further, the anti-infective drug also includes a pharmaceutically acceptable excipient.

Furthermore, the anti-infective drug is an oral preparation, an injectable preparation, or an external preparation.

The present invention has the following beneficial effects:

The present invention has found through research that sodium formate can significantly increase the sensitivity of a bacterium to an antibiotic, so that the antibiotic that is originally ineffective or inefficient against a pathogenic bacterium becomes effective or efficient, thereby killing the bacterium and achieving an anti-infective effect. Sodium formate and an antibiotic are further prepared into an anti-infective composition, so that on one hand, a significant anti-infective effect can be achieved under the condition of a low-concentration antibiotic, and on the other hand, the decreased use amount of the antibiotic can also significantly reduce the likelihood of a bacterium developing drug resistance; and sodium formate has been widely used in food and medicine with high safety.

### BRIEF DESCRIPTION OF THE DRAWINGS

FIG. 1 shows results of adding sodium formate to enhance the sensitivity of multidrug-resistant Escherichia coli to micronomicin in Embodiment 2, where A is a multidrug-resistant and carbapenem-resistant Escherichia coli series, and B is a multidrug-resistant but carbapenem-sensitive Escherichia coli series.

FIG. 2 shows experimental results of the effects of antibiotic concentrations (A), sodium formate concentrations (B), and treating time (C) on sodium formate enhancing the sensitivity of multidrug-resistant and carbapenem-resistant Escherichia coli to micronomicin in Embodiment 3.

FIG. 3 shows results of adding sodium formate to enhance the sensitivity of multiple bacteria to micronomicin in Embodiment 4, where A is Klebsiella pneumoniae, B is Staphylococcus aureus, C is Pseudomonas aeruginosa, and D is Vibrio, Edwardsiella tarda, and Escherichia coli.

FIG. 4 shows results of adding sodium formate to enhance the sensitivity of multidrug-resistant and carbapenem-resistant Escherichia coli to multiple antibiotics in Embodiment 5.

### DETAILED DESCRIPTION OF THE EMBODIMENTS

The present invention is further illustrated below in conjunction with the accompanying drawings and specific embodiments of the specification, but the embodiments do not impose any form of limitation on the present invention. Unless otherwise specified, the reagents, methods, and equipment used in the present invention are conventional reagents, methods, and equipment in the technical field.

Unless otherwise specified, the reagents and materials used in the following embodiments are commercially available.

### Embodiment 1 Bacterial Sample Preparation

Single bacterial colonies were selected and inoculated into a 5 mL LB medium, cultivated at 37° C. (clinically multidrug-resistant and carbapenem-resistant or multidrug-resistant but carbapenem-sensitive Escherichia coli, clinically multidrug-resistant and carbapenem-resistant or multidrug-resistant but carbapenem-sensitive Klebsiella pneumoniae, clinically multidrug-resistant and carbapenem-resistant or multidrug-resistant but carbapenem-sensitive Pseudomonas aeruginosa, methicillin-resistant Staphylococcus aureus, and sensitive bacteria for each type of bacteria) or 30° C. (Edwardsiella tarda and Vibrio) at 200 rpm for 16-24 hours; an appropriate amount of a bacterial solution was taken for centrifugation at 8000 rpm for 5 min to collect bacterial cells, the supernatant was removed, and the bacterial cells were washed with an equal volume of 0.85% normal saline for 3 times and suspended in M9 medium (containing 10 mM proline, 2 mM MgSO₄. 0.1 mM CaCl₂)), and a base bacterial solution was cultured with M9 to an OD₆₀₀ concentration of 0.6 and divided into 5 mL test tubes for later use.

### Embodiment 2 Sodium Formate can Enhance the Sensitivity of Multidrug-Resistant Escherichia coli, Including Carbapenem Antibiotic-Resistant Ones, to Micronomicin

Thirty-six clinical multidrug-resistant Escherichia coli samples were prepared according to Embodiment 1, of which 18 were clinical multidrug-resistant and carbapenem-resistant Escherichia coli (abbreviated as CR-EC) and 18 were clinical multidrug-resistant but carbapenem-sensitive Escherichia coli (abbreviated as MDR-EC). Each bacterial strain was divided into 4 groups. i.e., M9 medium control group, sodium formate group, micronomicin group, and micronomicin+sodium formate group; a concentration of micronomicin was 60 μg/mL (micrograms/mL), and a concentration of sodium formate was 20 mM. Each group had three biological replicates. After adding the corresponding drugs, each group was incubated in a shaking table at 37° C. and 200 rpm for 10 hours. Then 100 μL was taken and a serial dilution method was adopted, and 10 μL was taken respectively for dot plate counting. The number of bacteria was counted. Data on the number of bacterial colonies between 20 and 200 can be used for statistical analysis. The survival rate of bacteria after each treatment was the number of bacteria after 10 hours of sample treatment/the number of bacteria in the sample of the control group×100%.

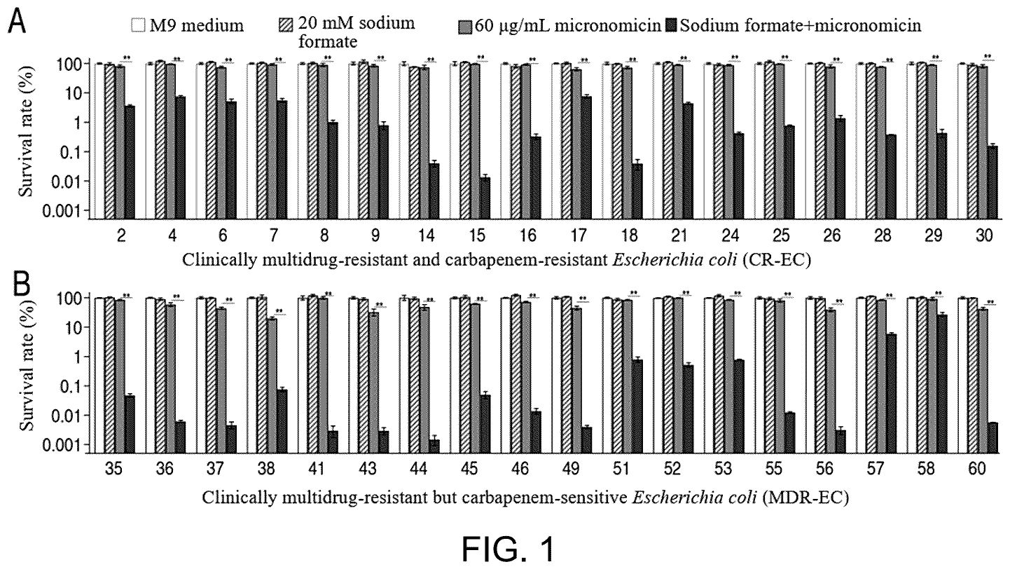

The results were illustrated in FIG. 1, which showed that the addition of 20 mM sodium formate alone has no killing effect on bacteria.

For clinically multidrug-resistant and carbapenem-resistant Escherichia coli, when only 60 μg/mL micronomicin antibiotic was added, all strains had survival rates of 67-97% (of which 5 strains had survival rates greater than 90%. 7 strains had survival rates greater than 80%. 5 strains had survival rates greater than 70%, and only 1 strain had a survival rate of 64%); however, after adding 20 mM sodium formate on the basis of this concentration of the antibiotic, the survival rate of strains decreased significantly, only 0.01%-7.36%, and the sensitivity was improved by 8.37-7250 times (A in FIG. 1).

For clinically multidrug-resistant but carbapenem-sensitive Escherichia coli, when only 60 μg/mL micronomicin antibiotic was added, only 10 strains had survival rates of 62% to 99% (of which 3 strains had survival rates greater than 90%. 5 strains had survival rates greater than 80%. 1 strain had a survival rate greater than 70%. 1 strain had a survival rate of 62%), and the remaining 8 strains had survival rates less than 60% (of which 1 strain had a survival rate greater than 50%. 4 strains had survival rates greater than 40%. 2 strains had survival rates greater than 30%, and 1 strain had a survival rate of 20%); adding sodium formate on the basis of this concentration of the antibiotic, the survival rate of bacteria was 0.01%-7.56%, and the sensitivity was improved by 3.32-33047 times, with the sensitivity of 5 bacterial strains being improved by 12000-33000 times (B in FIG. 1).

From the above results, it can be seen that sodium formate can not only significantly increase the sensitivity of clinically multidrug-resistant Escherichia coli to micronomicin, but also significantly enhance the sensitivity of clinically multidrug-resistant and carbapenem-resistant Escherichia coli to micronomicin, and the enhancing effect of carbapenem-sensitive bacteria was significantly better than that of carbapenem-resistant bacteria.

### Embodiment 3 Sodium Formate Enhancing the Sensitivity of Multidrug-Resistant Escherichia coli to Micronomicin was Dependent on Antibiotic Concentrations, Sodium Formate Concentrations, and Treating Time

Taking clinically multidrug-resistant and carbapenem-resistant Escherichia coli strain No. 28 as an example, adding different concentrations of sodium formate or different concentrations of antibiotics and under different treating times, the effect of improving the sensitivity of multidrug-resistant Escherichia coli to micronomicin was studied in depth.

### 3.1 Sodium Formate Enhancing the Sensitivity of Drug-Resistant Bacteria had an Antibiotic Concentration Gradient Effect

To understand how sodium formate increased the sensitivity of bacteria to micronomicin at different antibiotic concentrations, on the basis of exogenous addition of 20 mM sodium formate, 30-480 μg/mL (several concentrations of) micronomicin was added to treat bacteria. No antibiotic addition was used as a control, and live bacterial counts were performed to compare the survival rates of bacteria with and without the addition of sodium formate at the same antibiotic concentration after 10 hours.

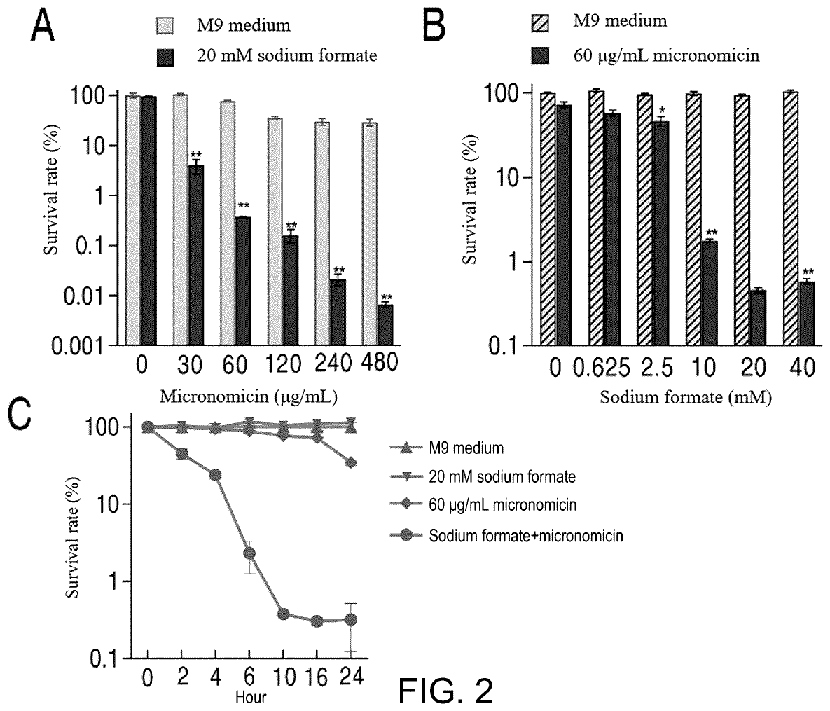

The result was shown as A in FIG. 2. It can be seen from the figure that under the premise of adding 20 mM sodium formate, as the concentration of micronomicin increased, its bactericidal efficiency against drug-resistant bacteria was improved more significantly, especially when adding 480 μg/mL micronomicin antibiotic, the bactericidal efficiency can be increased by nearly 4351 times. The specific situation was as follows: after adding 20 mM sodium formate, when the concentration of micronomicin was 30 μg/mL, the bactericidal efficiency of drug-resistant bacteria was improved by 26.73 times (the survival rate dropped from 100% without adding to 3.96% after adding), when the concentration of micronomicin was 60 μg/mL, the bactericidal efficiency of drug-resistant bacteria was improved to 203 times (the survival rate dropped from 77.37% without adding to 0.38% after adding), when the concentration of micronomicin was 120, 240 and 480 micrograms/milliliter in sequence, the bactericidal efficiency of drug-resistant bacteria was improved by 223 times (the survival rate dropped from 35.87% without adding to 0.16% after adding), 1395 times (the survival rate dropped from 29.77% without adding to 0.02% after adding) and 4351 times (the survival rate dropped from 29.01% to 0.006% after adding), respectively.

### 3.2 Sodium Formate Enhancing the Sensitivity of Drug-Resistant Bacteria had a Sodium Formate Concentration Gradient Effect

To investigate whether there was a gradient effect between the concentration of sodium formate and the bactericidal efficiency, as well as its optimal bactericidal concentration, different concentrations of sodium formate (0.625 mM-40 mM) were added on the basis of 60 μg/mL micronomicin addition for 10 hours, then live bacteria were counted, and the survival rate was calculated according to a formula as the following: the number of live bacteria with different concentrations of sodium formate addition/the number of live bacteria without sodium formate addition×100%.

The result was shown as B in FIG. 2. It can be seen from the figure that the bacterial survival rate of the control group (i.e., without sodium formate addition) was 72.3%. However, as the concentration of sodium formate added increased, the bacterial survival rate decreased from 57.69% to 0.47%, and the bactericidal efficiency was enhanced from 1.38 times to 203 times. It was worth noting that when the concentration of sodium formate was 40 mM, the bactericidal effect was weakened instead.

### 3.3 Sodium Formate Enhancing the Sensitivity of Drug-Resistant Bacteria had a Time Effect

Further research was conducted on adding 20 mM sodium formate and 60 μg/mL micronomicin and counting live bacteria at different time, and the relationship between its bactericidal efficiency and time was observed.

The result was shown as C in FIG. 2. It can be seen from the figure that the number of live bacteria of drug-resistant bacteria remained basically unchanged at 1-4 hours when only micronomicin was added without sodium formate addition; subsequently, there was a decrease, with a 6-hour survival rate of 87.96%, a 10-hour survival rate of 77.37%, and a 16-hour survival rate of 72.72%; it decreased significantly to 34.64% at 24 hours. However, when sodium formate was added at the same time as micronomicin, the number of live bacteria immediately decreased, the 2-4 hour survival rate decreased to 45.31% and 23.81%, and the sensitivity was improved by 2.15 times and 3.97 times; moreover, the number of live bacteria decreased obviously when the treating time was prolonged. Especially at 6-10 hours, the survival rate significantly decreased, with a 6-hour survival rate of 2.31% (sensitivity increased by 38 times) and a 10 hour survival rate of 0.35% (sensitivity increased by 203 times). After 16 hours, there was no significant increase in the bactericidal effect.

### Embodiment 4 Sodium Formate Enhanced the Sensitivity of Multiple Bacteria to Micronomicin

Multiple bacterial samples were prepared according to Embodiment 1. Bacterial strains included clinically multidrug-resistant carbapenem-resistant (abbreviated as CR-KP, strains No. 4 and 23) and multidrug-resistant carbapenem-sensitive Klebsiella pneumoniae (abbreviated as MDR-KP, strain No. 42), clinically multidrug-resistant carbapenem-resistant (abbreviated as CR-PA, strains No. A2, C3 and D2) and multidrug-resistant carbapenem-sensitive Pseudomonas aeruginosa (abbreviated as MDR-PA, strain No. 3), methicillin-resistant Staphylococcus aureus MRSA, as well as sensitive bacteria for each type of bacteria (Klebsiella pneumoniae ATCC700603, Staphylococcus aureus MSSA. Pseudomonas aeruginosa PA-S), Escherichia coli K12 and ECO-S16; Edwardsiella tarda (EIB202 and ET17), and Vibrio (ZNV4 and ZNV10, obtained from seafood isolation and identified as Vibrio genus through 16sRNA identification).

Each bacterial strain was divided into 4 groups, i.e., M9 medium control group, 20 mM sodium formate group, micronomicin group, and micronomicin+sodium formate group. Each bacterial strain was added with different concentrations of micronomicin, and the specific amount of addition was shown in Table 1. There were three biological replicates.

TABLE 1
Doses of micronomicin antibiotic used for different strains

| | Micronomicin | | Micronomicin | | Micronomicin |
|---|---|---|---|---|---|
| Strain | (μg/mL) | Strain | (μg/mL) | Strain | (μg/mL) |
| CR-KP4 | 1.5 | CR-PA-A2 | 40 | ZNV4 | 5 |
| CR-KP23 | 60 | CR-PA-C3 | 5 | ZNV10 | 5 |
| MDR-KP42 | 1 | CR-PA-D2 | 0.5 | EIB202 | 30 |
| ATCC700603 | 60 | MDR-PA3 | 2 | ET17 | 30 |
| MRSA | 10 | PA-S1 | 2 | K12 | 1 |
| MSSA | 1 | | | ECO-S61 | 20 |

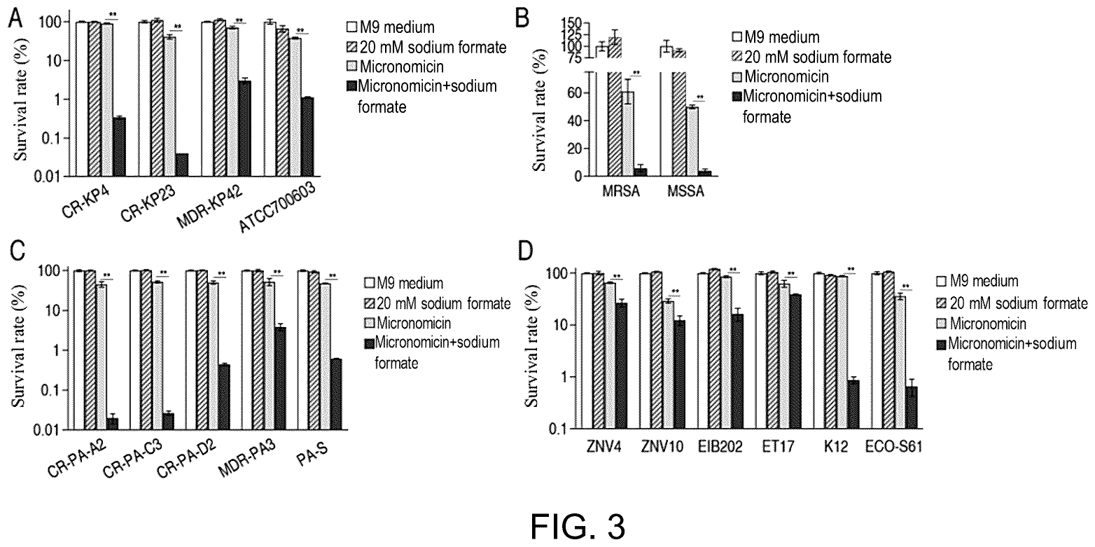

The results were shown in FIG. 3. It can be seen from the figure that on the basis of adding sodium formate, the sensitivity of these bacteria to micronomicin was generally improved significantly. This indicated that sodium formate can significantly increase the sensitivity of these bacteria to micronomicin.

### Embodiment 5 Sodium Formate Enhanced the Sensitivity of Multidrug-Resistant Escherichia coli to Other Antibiotics

To investigate whether the addition of sodium formate is effective to antibiotics other than micronomicin in multidrug-resistant Escherichia coli, a clinical multidrug-resistant and carbapenem-resistant Escherichia coli sample No. 28 was prepared according to Embodiment 1, and 20 mM sodium formate and different kinds of antibiotics were added, respectively. The concentration of antibiotics added was shown in Table 2. After 10 hours of action, the number of live bacteria was counted, and the survival rate of the bacteria with different antibiotics was calculated after adding sodium formate.

TABLE 2
Antibiotics and their doses used

| Antibiotic | Concentration (μg/mL) |
|---|---|
| Gentamicin | 120 |
| Amikacin | 120 |
| Meropenem | 80 |
| Imipenem | 80 |
| Ceftazidime | 200 |
| Cefoperazone-sulbactam | 200 |
| Balofloxacin | 40 |
| Ciprofloxacin | 40 |

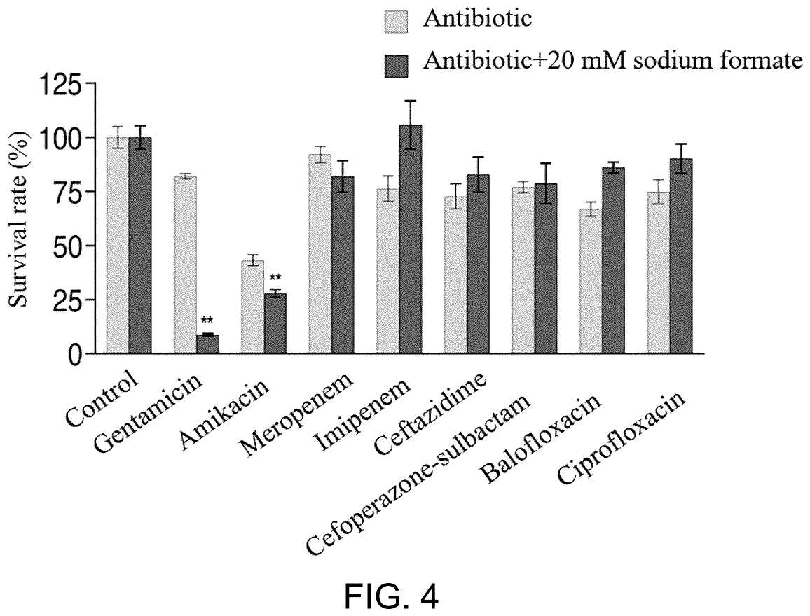

The results were shown in FIG. 4. It can be seen from the figure that sodium formate can only increase the sensitivity of clinically multidrug-resistant and carbapenem-resistant Escherichia coli strains to gentamicin and amikacin, with sensitivity increases of 9.55 and 1.5 times, respectively, while it had no effect on other antibiotics.

The above embodiments are the preferred embodiments of the present invention, but the implementations of the present invention are not limited by the above embodiments. Any other changes, modifications, substitutions, combinations, or simplifications that do not deviate from the spirit and principles of the present invention should be equivalent substitution methods and are included in the scope of protection of the present invention.

1. (canceled)

2. (canceled)

3. (canceled)

4. (canceled)

5. (canceled)

6. (canceled)

7. An anti-infective composition, comprising sodium formate and an aminoglycoside antibiotic.

8. The composition according to claim 7, wherein the aminoglycoside antibiotic is one or more of micronomicin, gentamicin, and amikacin.

9. The composition according to claim 7, wherein a mass ratio of the sodium formate to the antibiotic is (0.088-90.67):1.

10. An anti-infective drug, comprising the anti-infective composition according to claim 7.

11. The composition according to claim 8, wherein a mass ratio of the sodium formate to the antibiotic is (0.088-90.67):1.

12. An anti-infective drug, wherein the anti-infective drug comprises the anti-infective composition according to claim 8.

13. An anti-infective drug, wherein the anti-infective drug comprises the anti-infective composition according to claim 9.

14. An anti-infective drug, wherein the anti-infective drug comprises the anti-infective composition according to claim 11.

15. An anti-infection method, comprising:
combining sodium formate with an antibiotic, to enhance sensitivity of a bacterium to the antibiotic by a synergistic effect of the sodium formate and the antibiotic, thereby killing the bacterium and achieving an anti-infective effect.

16. The method according to claim 15, wherein the antibiotic is an aminoglycoside antibiotic.

17. The method according to claim 16, wherein the aminoglycoside antibiotic is one or more of micronomicin, gentamicin, and amikacin.

18. The method according to claim 15, wherein the bacterium is an antibiotic-sensitive or clinically drug-resistant Gram-negative bacterium.

19. The method according to claim 18, wherein the bacterium is Escherichia coli, Klebsiella pneumoniae, Pseudomonas aeruginosa, Edwardsiella tarda, Vibrio, or Staphylococcus aureus.
